(19) United States
(12) Patent Application Publication
(10) Pub. No.: US 2010/0331632 A1
(43) Pub. Date: Dec. 30, 2010

(54) WIRELESS POLYSOMNOGRAPHY SYSTEM

(76) Inventor: Chang-An CHOU, Taipei (TW)

Correspondence Address: Chang-An Chou, 3F, No. 100, Sec. 3. Mingsheng E. Rd., Taipei, 105, TW

(21) Appl. No.: 12/824,243

(22) Filed: Jun. 28, 2010

(30) Foreign Application Priority Data
Jun. 29, 2009 (CN) 200910087574.6

(51) Int. Cl. A61B 5/00

(52) U.S. Cl. 600/301

(57) ABSTRACT

A distributed wireless polysomnography (PSG) system is provided. The system includes plural wireless physiological signal acquiring devices and a base station, in which the base station is wirelessly and bi-directionally communicates with the wireless physiological signal acquiring devices. During PSG examination, the wireless physiological signal acquiring devices are worn by a patient and the base station is connected to a remote computer device via a network. Then, each of the wireless physiological signal acquiring devices acquires physiological signals through at least a sensing element connected thereto and/or built therein, and the acquired physiological signals are wirelessly transmitted, in real time, to the base station and then to, via the network, the remote computer device, so as to achieve a real time monitoring of the patient's physiological signals during sleep. Moreover, the base station is capable of executing at least one of configuring the wireless physiological signal acquiring devices, controlling the operations of the wireless physiological signal acquiring devices, displaying the physiological signals acquired by and transmitted from the wireless physiological signal acquiring devices, and indicating the statuses of the wireless physiological signal acquiring devices during the operations.

### FIELD OF THE INVENTION

The present invention is related to a wireless polysomnography (PSG) system, and more particularly to a distributed multi-channel physiological signal monitoring system used during sleep which can provide higher mobility and comfortability to the patient, as well as an improved operation convenience to the operator.

### BACKGROUND OF THE INVENTION

Nowadays, people have developed higher standards and demands with respect to the sleep quality. Therefore, there are more and more researches have focused on sleep, in which the polysomnography (PSG) system plays an important role.

Usually, the PSG system acquires physiological signals of respiration (including air flow and respiratory efforts), snoring, oxygen saturation, ECG, EEG, EMG (including facial and limb), EOG, and body position, etc. And, through analyzing the related physiological signals, the sleep quality can be revealed and the sleep disorder can be found if any.

The conventional PSG system restricts the patient's motion seriously since there are considerable quantity of wires connected between the sensors/electrodes attached on the patient and the receiving device aside, and this situation may significantly reduce the patient's motivation for accepting the examination.

Afterward, the ambulatory PSG system has been developed, in which the volume of the receiving device is reduced for being able to be carried by the patient and thus shorten the connecting wires to the electrodes/sensors, thereby providing the patient a more comfortable using experience. However, owing to the large amount of physiological signals that should be acquired, the ambulatory PSG system still will have a considerable volume.

Moreover, for a PSG examination, in addition to physiological signal acquisition, the real-time monitoring is also important. For example, the operator (sleep technician and/or doctor) has to check the accuracy and reliability of electrode/sensor installation at the beginning of monitoring (the calibration process), and also sometimes need to mark special events during sleep. Hence, normally, the ambulatory system still has to connect to a monitoring device aside the operator. Generally, the connection to the monitoring device can be achieved in a wired or wireless manner. If it adopts the wireless manner, the patient can experience a better mobility than the restrictive wired connection.

However, since the monitoring device which is necessary for executing the calibration process and the event marking is always located in a room different from the patient, there actually exists the inconvenience of travelling to another room for adjusting the electrodes/sensors.

Therefore, how to develop a superior architecture for the PSG system for reducing the burden and simultaneously the wiring complexity on the patient has become a significant issue.

And, it is also important to provide a PSG system which provides higher mobility and comfortability to the patient, as well as an improved operation procedure to the operator.

The object of the present invention is to provide a PSG system which employs a distributed architecture to spread out the weight burden on the patient, and also, to provide system flexibility and scalability.

Another object of the present invention is to provide a distributed PSG system which adopts the wireless technology to provide the patient the mobility and also the operator the convenience during monitoring.

### SUMMARY OF THE INVENTION

According to one aspect of the present invention, a distributed wireless polysomnography (PSG) system including plural wireless physiological signal acquiring devices and a base station is provided, and in this system, the base station can wirelessly and bi-directionally communicate with the wireless physiological signal acquiring devices. As proceeding the PSG examination, the wireless physiological signal acquiring devices are worn by a patient and the base station is connected to a remote computer device via a network. Then, each of the wireless physiological signal acquiring devices acquires physiological signals through at least a sensing element connected thereto and/or built therein, and the acquired physiological signals are wirelessly transmitted, in real time, to the base station and then to, via the network, the remote computer device, so as to achieve a real time monitoring of the patient's physiological signals during sleep. Moreover, the base station is capable of executing at least one of configuring the wireless physiological signal acquiring devices, controlling the operations of the wireless physiological signal acquiring devices, displaying the physiological signals acquired by and transmitted from the wireless physiological signal acquiring devices, and indicating the statuses of the wireless physiological signal acquiring devices during the operations.

Moreover, the wireless physiological signal acquiring devices can be equipped with memories to store the acquired physiological signals, so as to provide a data resource for analysis, for example, it can be that each physiological signal acquiring device includes a memory for data storage, and after the PSG examination, the data from plural devices are combined to provide a complete PSG data. Besides, by wireless transmitting, the data also can be stored in the base station, and/or the remote computer device, e.g., a flash memory or heard disk, without limitation. Here, the memories in each wireless physiological signal acquiring device and/or the base station can be implemented to be removable, such as, a memory card.

According to the present invention, the base station can be utilized to execute an impedance check and a waveform display of the acquired physiological signals, so that the operator (technician/doctor) can immediately adjust the electrode/sensor installation right after the calibration process, and the base station can further provide the operator a status indication of the wireless physiological signal acquiring device, e.g., the attaching situation on the patient's body surface. Alternatively, the operator also can control/configure the wireless physiological signal acquiring devices through the remote computer device, by means of a software embedded therein and the base station.

Furthermore, the base station can be implemented to analyze the received physiological signals and/or to determine a physiological state of the patient based thereon, so that if the physiological state matches to a preset condition, the base station can send out a warning signal to notify the operator in front of the remote computer device, wherein the preset condition can be configured by the operator through the base station and/or the remote computer device.

In a preferred embodiment of the present invention, an adapting device including a network interface and a power interface is further employed to connect the base station to the network and also the power source. Therefore, as the base station is equipped with a battery, it can be separately operated from the adapting device. Plus, in this case, it is also a reasonable selection for the adapting device to provide a wired network connection for better reliability. However, t should be noted that the network for connecting the base station and/or the adapting device to the remote computer device can be implemented to be a wireless or wired data network, such as, TCP/IP, without limitation. More advantageously, the adapting device can further include other communication interfaces, such as, RS232, for expanding the functionality of the base station. And, the adapting device can be implemented to have a dock structure for receiving the base station.

For facilitating the analysis and diagnosis of the PSG examination, it is preferable that the base station is implemented to include a light sensor for sensing the changes of light in the environment, and at least one of the wireless physiological signal acquiring devices is implemented to include an event marker for being pressed by the patient as a special event occurs.

Each of the wireless physiological signal acquiring devices is implemented to equip with a battery for providing the operation power, and the sensing element of each device can be one or more selected from a group consisting of: a flow sensor, a thermistor, a snore sensor, respiratory effort belts, EEG electrodes, EOG electrodes, ECG electrodes, EMG electrodes, an oximeter, and a position sensor, wherein the position sensor can be built in at least one of the wireless physiological signal acquiring devices.

In another embodiment of the present invention, the sensing elements can be connected to the wireless physiological signal acquiring devices via a connection mechanism, e.g., a flat-typed connector for reducing the space occupied by numerous connectors in the PSG system. And further, it is also advantageously that the connection mechanism can be implemented to connect multiple sensing elements to one wireless physiological signal acquiring device at the same time, so as to contribute to the reduction of wiring complexity.

Consequently, the PSG system of the present invention provides a more ergonomic arrangement on the patient and also the independency of each physiological signal acquiring device, by utilizing a distributed architecture which separates the conventional single PSG device into multiple smaller physiological signal acquiring devices as well as the wireless technology. Accordingly, the wires from the physiological signal acquiring devices to the base station can be eliminated and also the wires to the sensing elements can be shortened, so as to reduce the wiring complexity; and further, the quantity of the devices and the constitution of the system can be varied based on different demands, for example, one or some of the devices can be used to acquire physiological signals individually or cooperatively instead of employing the whole system, and one or more additional device(s) can be joined to the system to satisfy extra requirements, so as to improve the using flexibility and the system scalability. And further, by employing the memory for data storage, the device(s) even can be taken home by the patient, and by further using the base station (and the adapting device), a remote real-time monitoring also can be achieved. Moreover, the base station is implemented to wirelessly control and configure all the physiological signal acquiring devices, which provides the operator a convenient calibration process, such as, impedance check and waveform confirmation, aside the patient after installing the electrodes/sensors. Furthermore, the network-based system architecture allows the remote computer device to simultaneously receive data from multiple basic base stations and thus monitor multiple patients in real time.

### BRIEF DESCRIPTION OF THE DRAWINGS

A more detailed understanding of the invention may be had from the following description of preferred embodiments, given by way of example, and to be understood in conjunction with the accompanying drawings, wherein:

FIG. 1 is a schematic view showing a wireless polysomnography system in a preferred embodiment of the present invention;

FIG. 2 is a schematic view showing a wireless polysomnography system in another preferred embodiment of the present invention;

FIG. 3 is a schematic view showing an exemplary application of FIG. 1; and

FIG. 4 is a schematic view showing an exemplary application of FIG. 2.

### DETAILED DESCRIPTION OF THE PREFERRED EMBODIMENTS

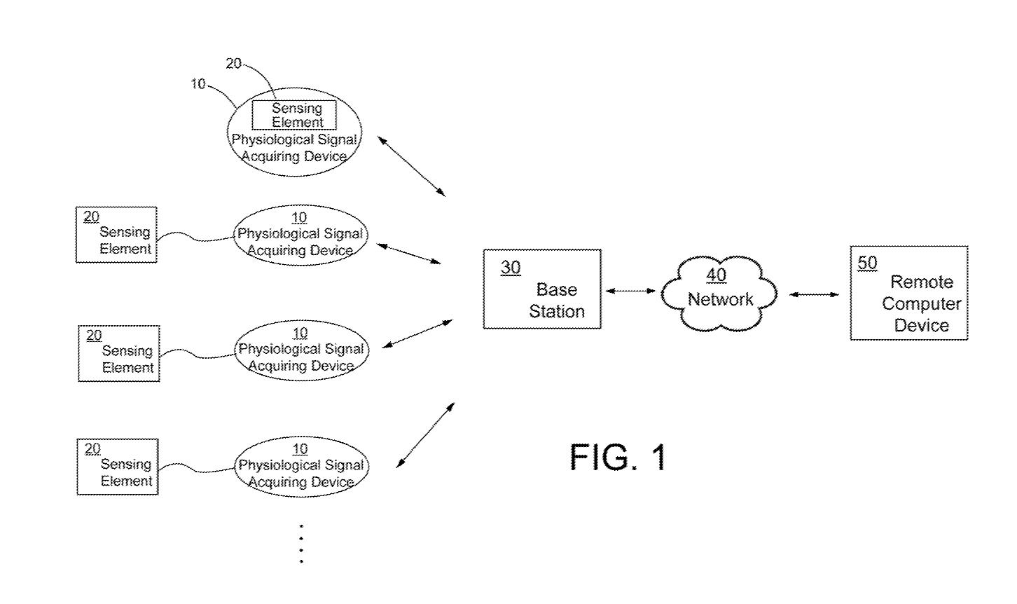

Please refer to FIG. 1, which is a schematic view showing a wireless polysomnography (PSG) system according to the present invention. The PSG system includes plural wireless physiological signal acquiring devices 10 and a base station 30. The plural wireless physiological signal acquiring devices 10 should be worn by a patient and each will have at least a sensing element 20 connected thereto and/or built therein for acquiring physiological signals. The base station 30 is connected to a remote computer device 50 via a network 40. During the PSG examination, the acquired physiological signals will be wirelessly transmitted by the device 10 to the base station 30, and then to a remote computer device 50, the base station 30, via the network, thereby achieving a real-time monitoring.

Here, the sensing elements 20 can be implemented to be electrodes and/or sensors, such as, EEG electrodes, EOG electrodes, ECG electrodes, EMG electrodes, a flow sensor, a thermistor, a snore sensor, respiratory belts, an oximeter, and/or a position sensor.

Since the ambulatory PSG system should be worn by the patient during sleep, it is better to minimize the volume thereof for providing the patient a maximum convenience and comfortability. However, as known, the more the number of acquired physiological signals, the larger the volume of the device used for acquiring these physiological signals. Therefore, since a complete PSG system depends on the comprehensive physiological signal acquisitions, there actually exists a limitation in minimizing the volume thereof.

For solving this problem, the present invention divides the needed physiological signals into several groups, such as, but not limited, physiological signals acquired around the head, physiological signals acquired on the torso, physiological signals acquired on the limbs, and physiological signals related to respiration, and each group of physiological signal acquisitions is connected to one device (including related circuitry therein and connectors) smaller than the conventional one, so as to form a PSG system with plural physiological signal acquiring devices. Accordingly, each device can be mounted at a position near the acquired physiological signals, for example, the device for EEG acquisition can be mounted on the forehead, the device for oxygen saturation detection can be worn on the wrist, and the device for detecting respiratory effort can be directly mounted on the thoracic belt and/or the abdominal belt. Here, it should be noted that the grouping of physiological signals can be varied in accordance with different demands, e.g., it can be grouped based on the position, function, or practicability of each physiological signal, without limitation.

Because the plural physiological signal acquiring devices are designed to be independent of one another without wire connection, during the physiological signal acquisition, only wirings used for connecting the sensing elements 20 to the devices 10 will be spread on the patient's body surface. Plus, each device 10 is also equipped with battery and wireless module, so that the wirings for power supply and data transmission are also eliminated.

As a result, through this distributed architecture, first, the weight of the conventional device can be separated and carried by different portions of the patient's body for spreading out the burden on the patient and thus more conforming to the ergonomics, and further, the wires between the sensing elements 20 and the wireless physiological signal acquiring devices 10 can be shortened or eliminated for reducing the wiring complexity and also the cost.

Besides, for further reducing the volume of the physiological signal acquiring device 10, the connection mechanism between the sensing element(s) 20 and the device 10 can be implemented to be a flat-typed connector for reducing the large space occupied by the conventional electrode/sensor connectors. Moreover, the connection mechanism can be designed to simultaneously connect multiple sensing elements 20 to one device 10, which not only can reduce the wiring complexity but also can simplify the electrode/sensor installation process.

In addition to reducing the burden and complexity on the patient's body, the requirements of the operator (technician/doctor) are also considered in the present invention. Under the distributed architecture of the present invention, it might become more complicated and inconvenient for the operator to configure and operate multiple physiological signal acquiring devices 10 in one patient's PSG examination, so that the present invention further provides a base station 30 for simultaneously controlling plural devices 10, such as, initiating/terminating physiological signal acquisitions and/or configuring parameters. That is, the wireless communications between the base station 30 and plural devices 10 are bi-directionally. Accordingly, the operation interface on each device 10 can be simplified to, for example, a button for power on/off and/or a status indicator, so as to further reduce the volume thereof.

In a preferred embodiment, instead of operating in front of the remote computer device in another room, by directly operating the base station 30, the operator can perform an impedance check aside the patient. This is a great improvement in convenience. Since the accuracy and reliability of electrode installation are the foundation of success physiological signal acquisition, the confirmation procedure is necessary and important, so that if the operator can check the signals through the base station 30 and immediately adjust the electrode(s) instead of instructing from another room, the operation procedure can be significantly simplified and improved.

In another preferred embodiment, the base station 30 can be implemented to display the waveforms of the acquired physiological signals, so that the operator can conveniently check the acquired physiological signals (from sensors and electrodes) through the base station 30. Moreover, the base station 30 also can be implemented to display the statuses of the devices 10, e.g., the power level, the attaching condition to the patient, and the quality of wireless transmission.

In addition to parameter configuration and installation confirmation, in still another preferred embodiment, the base station 30 also can provide a warning function during the physiological signal acquisition. The base station 30 can be implemented to analyze the physiological signals received from the devices 10 and compare the analysis result to a preset condition which can represent an abnormal physiological condition, e.g., heart rate, body temperature, and/or the number of respiration per minute, so as to output a warning/rescue signal as the preset condition is matched. Note that the preset condition can be varied from patient to patient without limitation. More advantageously, at least one of the wireless physiological signal acquiring devices 10 can be provided with an event marker for being pressed by the patient as a particular physiological condition occurs, so as to facilitate the doctor's analysis and diagnosis.

Besides, a light sensor (not shown) also can be provided in the base station 30 for sensing the change of light in the environment, which also can contribute to the analysis and diagnosis.

In the present invention, the base station 30 is connected to a remote computer device 50 via a network 40, so that the wirelessly received physiological signals can be transmitted to the remote computer device 50 in real time. Because the transmission is achieved by a network interface, one computer device can easily receive data from multiple base stations via a hub, which is different from the conventional situation that one computer only can receive data from one PSG device. This is especially beneficial to the sleep center which might have multiple PSG monitoring in processing at one night. Through the PSG system of the present invention, each base station 30 placed in each room can transmit the acquired physiological signals of different patients back to the computer device in front of the operator via the network 40. Here, because the base station 30 is placed in the patient's room, it can ensure that the devices 10 are located in the wireless coverage of the base station 30. Therefore, without setting up as many computer devices as the quantity of the PSG systems, the cost for establishing the sleep center can be reduced. Besides, owing to the popularity of network, it is also possible to utilize the original network system in the hospital for further reducing the cost and the complexity for arranging the network cable. It should be noted that the network 40 can be implemented to be wired or wireless, that is, the base station 30 can be connected to the remote computer device 50 in a wired or wireless manner, without limitation.

Moreover, the base station 30 can be powered by a battery or connecting to a power source. When the usage of battery cooperating with the wireless network 40, the base station 30 can be operated in a cordless manner. However, the power consumption might be an issue since the real-time monitoring will last for all night. Therefore, one way is to increase the volume of the battery, and another is to employ a power cord for the base station. And, if the base station has already connected with a power cord, then it will be reasonable to also utilize the wired network to seek for more reliable data transmission. But, the arrangement can be varied in accordance with real demands, there is no limitation.

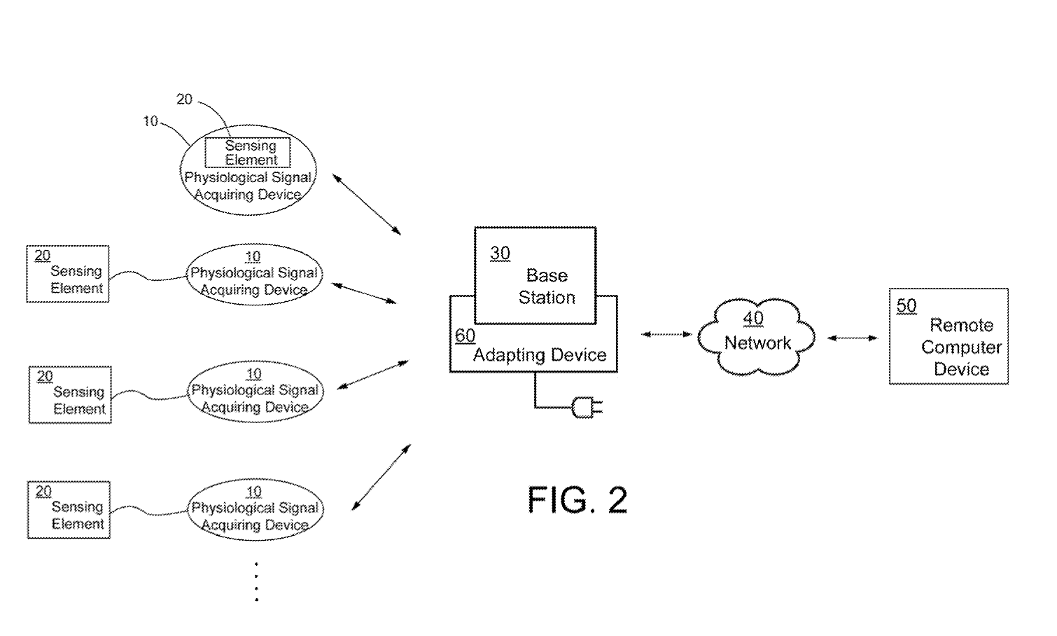

Accordingly, in a preferred embodiment, an adapting device 60 is further employed to connect the base station 30 to the network 40 and a power source, as shown in FIG. 2.

The adapting device 60 is implemented to have a network interface and a power interface as well as a port for connecting to the base station, a port for connecting to the network (and the remote computer device), and a port for connecting to the power source. In this case, the base station 30 is connected with the adapting device 60 for accessing the network 40, so as to transmit the received physiological signals from plural physiological signal acquiring devices 10 to the remote computer device 50. It is preferably that the base station 30 is simultaneously charged by the power source as being connected with the adapting device 60, that is, the base station 30 is equipped with a rechargeable battery and the port for connecting to the base station can achieve data and power transmissions at the same time. Accordingly, the base station 30 can leave the adapting device 60 for a short-term operation, such as, parameter configuration, impedance check, and/or system initiation/termination, without power shortage and being interfered by the power cord and the network cable. After completing, the base station 30 can be connected back to the adapting device 60 for obtaining the power supply and the networking capability the installation, and then, the patient falls asleep and the real-time monitoring starts. Since, during the real-time monitoring, it is rare that the operator has to go into the patient's room for further adjustment, it only has to ensure that the base station 30 stays at a position that can receive the physiological signals from the devices 10 worn by the patient, and then, the real-time monitoring can last for all night without interruption.

In the present invention, during the real-time monitoring, the physiological signals are sent out to the remote computer device 50 right after being received by the base station 30. That is, the real-time monitoring provides the newest data. Therefore, as employing the adapting device 60 in which the base station 30 might be operated separately therefrom to interrupt the real-time transmission, the real-time data display on the remote computer device 50 will stop during the separation, and then restart as they are reconnected.

Here, although the adapting device 60 is depicted to have a dock structure in FIG. 2 for receiving the base station 30 in a more convenient and stable way, the physical implementation of the adapting device can be varied in accordance with different demands without limitation.

Further, because the analysis and interpretation of the physiological signals have identical importance to the real-time monitoring, in the present PSG system, the acquired physiological signals also will be stored in the memory of the physiological signal acquiring device 10, the base station 30 and/or the remote computer device 50, such as, flash memory or hard disk. In a preferred embodiment, it can be implemented to be each wireless physiological signal acquiring device 10 is equipped with a memory for storing the acquired physiological signals. Then, after examination, the data from multiple devices 10 are combined to provide a complete PSG data for the technician/doctor to carry out further analysis and diagnosis. Here, the memory for storage also can be implemented to be removable, e.g., a memory card, so as to further facilitate the data access. Therefore, the employment of memory storage ensures the provision of data source used in further analysis and diagnosis.

Therefore, the present invention provides a different angle of view for the wireless PSG system which not only considers the patient's conformability, but also provides the operator a convenient using experience during physiological signal acquisition.

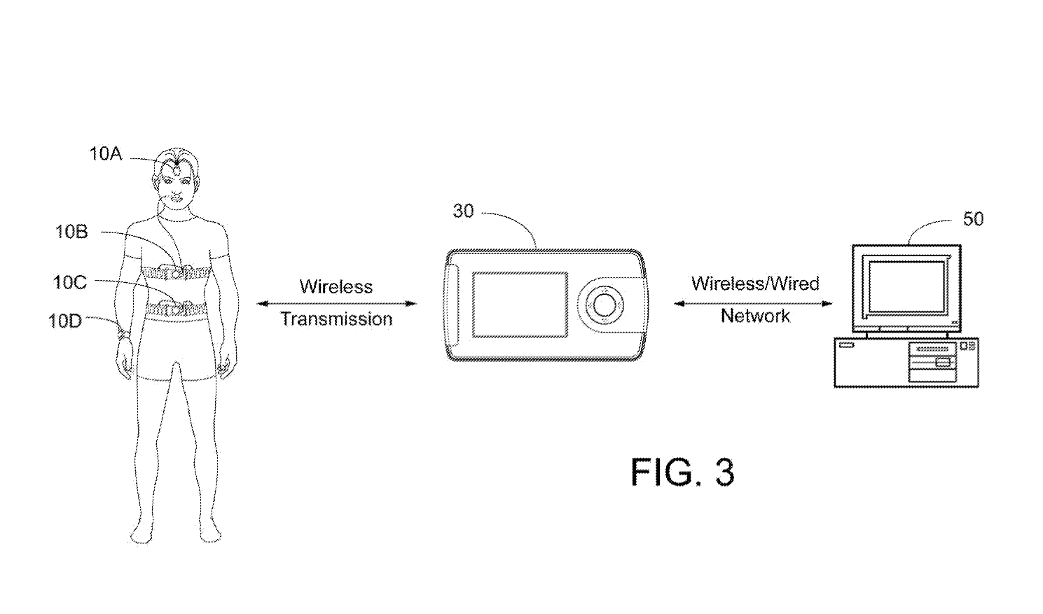

Now, please refer to FIG. 3. As shown, when proceeding a PSG examination, multiple wireless physiological signal devices 10 are worn by the patient. Here, the positions for placing the devices 10 are depended on the types of physiological signals acquired, for example, the EEG/EOG signal acquiring device 10A is place on the forehead, the oxygen saturation detection device 10D is worn on the wrist, and the respiratory signal acquiring devices 10B, 10C are placed on the respiratory belts (also can be place on the cheek). Then, the sensing elements can be fixed to the specific positions for physiological signal acquisitions.

It should be noted that only partial the physiological signal acquiring devices for a PSG examination are shown in FIG. 3. As known, a PSG system acquires a large amount of physiological signals, including, but not limited, EEG, EOG, air flow and thermal variations of respiration, snoring, facial EMG (chin and cheek), ECG, respiratory effort, limb movement, and body position etc. Besides, the sensing elements also can be implemented into different types and/or to have different installation manners, for example, the oximeter can be positioned on the forehead, ear or finger depending on the type thereof. And, the connection thereof to the wireless physiological signal acquiring device also can be varied based on the real needs, for example, the connection mechanism can be achieved by a flat-typed connector, and/or the acceleratory sensor can be built in the physiological signal device. There is no limitation.

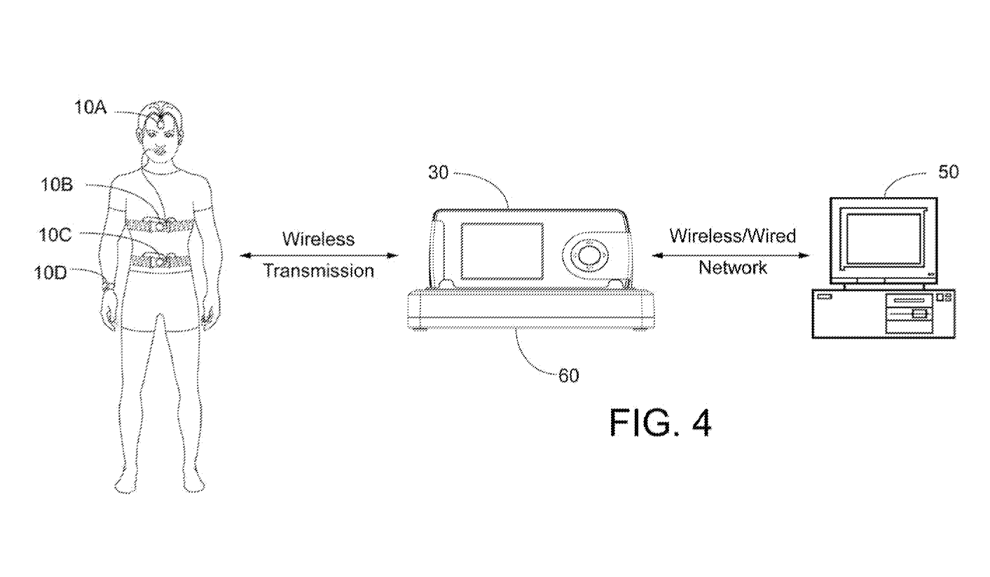

After the physiological signals are acquired, the acquired physiological signals are wirelessly transmitted by the wireless physiological signal acquiring devices 10 to the wireless base state 30, and then, to the remote computer device 50 by the base station 30 via the network 40, thereby achieving a physiological signal transmission for real-time monitoring. Alternatively, if the adapting device 60 is employed, as shown in FIG. 4, before arriving the remote computer device 50, the physiological signals are transmitted to the adapting device 60 in advance.

In the present invention, the communications are all bi-directional. Therefore, through a proper design of software, the operator can directly control (e.g., initiate/terminate and/or configure) each PSG examination in different rooms by operating the remote computer device. The command will arrive the base station 30 via the network 40 (and the adapting device 60) and then to the physiological signal acquiring devices 10 for controlling. After that, the physiological signal acquiring devices 10 restart to acquire and wirelessly transmit the physiological signals, and via the base station 30 (and the adapting device 60) and the network 40, the physiological signals arrive the remote computer device 50 again.

Advantageously, owing to the distributed architecture and the wireless technology, each physiological signal acquiring device 10 in the present invention can possess independency, so as to provide the usage flexibility for the system, both are not found in the prior arts.

Generally, the technician/doctor has to prepare several kinds of examination devices for different examination purposes, such as, for screening or for detailed examination during sleep. In the present invention, each physiological signal acquiring device can be operated independently or cooperated with one another without quantity limitation, so that for screening purpose, the doctor can only select one or two physiological signal acquiring devices essential for sleep diagnosis (such as, respiration and snoring) to carry out partial PSG examination, so as to provide a reduced weight on the patient, compared with the conventional large-volume and indivisible PSG system. Then, after screening, the patients who do have sleep disorders can use the full-function PSG system by employing all the physiological signal acquiring devices, for further confirmation. Therefore, the present invention provides a great flexibility for satisfying different using requirements.

Furthermore, owing to the small volume, easy installation process and the memories for data storage, the patient even can take the device(s) home, thereby reducing the waiting list of the sleep center. Further, by using the base station and the network at the patient's home, a remote real-time monitoring also can be achieved.

More advantageously, the distributed architecture and the wireless technology of the present invention also provide the scalability for the PSG system. Similar to the partial usage of PSG system above, addition physiological signal acquisition device(s) also can be joined to the PSG system for satisfying extra requirements. For example, more EEG devices 10 can be joined to the system to acquire additional EEG signals for achieving a more detailed EEG examination, and this is also practicable for a multiple-lead ECG examination.

In other words, the present invention achieves a variable constitution of the PSG system through the independency of each physiological signal acquiring device. Therefore, by increasing/decreasing the quantity of devices and/or changing the types of devices, the composition of physiological signals to be acquired in the PSG system can be customized without limitation.

In addition, the distributed architecture also allows the present invention to be used in the non-sleep period since a PSG system acquires comprehensive physiological signals. The doctor/technician can take one physiological signal acquiring device or combine multiple needed types and quantities of devices to perform the examination, which is difficult to be achieved in the conventional one-device PSG system. Plus, owing to the memory equipped for data storage, it can be selected to or not to execute the real-time monitoring. Therefore, it is easy to apply the present PSG system to different fields of physiological examinations to save the cost.

It should be noted that in addition to each physiological signal acquiring device can be used independently, the multiple kinds of physiological signals acquired in each physiological signal acquiring device also can be enabled/disabled independently. That is, in each examination, through the base station 30 and/or the remote computer device 50, the operator can select and configure the type(s) and the quantity of physiological signals to be acquired without considering the grouping provided by the device.

In the aforesaid, the PSG system of the present invention utilize a distributed architecture, which separates the conventional single PSG device into multiple smaller physiological signal acquiring devices, as well as the wireless technology, to provide a more ergonomic arrangement on the patient and also the independency of each physiological signal acquiring device. Accordingly, the wires from the physiological signal acquiring devices to the base station can be eliminated and also the wires to the sensing elements can be shortened, so as to reduce the wiring complexity; and further, the quantity of the devices and the constitution of the system can be varied based on different demands, and one or more additional device(s) can be joined to the system to satisfy extra requirements, so as to provide the using flexibility and the system scalability. And further, by employing the memory for data storage, the device(s) even can be taken home by the patient, and by further accompanying the base station (and the adapting device), a remote real-time monitoring also can be achieved. Moreover, the base station is implemented to wirelessly control and configure the physiological signal acquiring devices so as to provide the operator a convenient calibration process, such as, impedance check and waveform confirmation, aside the patient after installing the electrodes/sensors. Furthermore, the network-based system architecture allows the remote computer device to simultaneously receive data from multiple basic base stations and thus monitor multiple patients in real time.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. A wireless polysomnography (PSG) system, comprising plural wireless physiological signal acquiring devices, and a base station,
wherein:
the base station wirelessly and bi-directionally communicates with the wireless physiological signal acquiring devices;
the wireless physiological signal acquiring devices are worn by a patient and the base station is connected to a remote computer device via a network; and
each of the wireless physiological signal acquiring devices has at least a sensing element connected thereto and/or built therein for acquiring physiological signals, and the acquired physiological signals are wirelessly transmitted, in real time, to the base station and then to, via the network, the remote computer device, so as to achieve a real time monitoring of the patient's physiological signals during sleep; and
wherein:
the base station is capable of executing at least one of:
configuring the wireless physiological signal acquiring devices;
controlling the operations of the wireless physiological signal acquiring devices;
displaying the physiological signals acquired by and transmitted from the wireless physiological signal acquiring devices; and
indicating the statuses of the wireless physiological signal acquiring devices during the operations.

2. The system as claimed in claim 1, wherein each of the wireless physiological signal acquiring devices further comprises a memory for storing the acquired physiological signals to be used in an analysis process.

3. The system as claimed in claim 2, wherein the stored data from the wireless physiological signal acquiring devices are combined in the analysis process.

4. The system as claimed in claim 2, wherein the memory for data storage is implemented to be removable.

5. The system as claimed in claim 1, wherein the acquired physiological signals are stored in the base station and/or the remote computer device.

6. The system as claimed in claim 1, wherein at least one of the wireless physiological signal acquiring devices is implemented to include an event marker.

7. The system as claimed in claim 1, wherein the operations of the wireless physiological signal acquiring devices comprise executing an impedance check.

8. The system as claimed in claim 1, wherein the statuses of the wireless physiological signal acquiring devices comprise the attachments to the patient's body surface.

9. The system as claimed in claim 1, further comprising an adapting device, having a network interface and a power interface, connected the base station.

10. The system as claimed in claim 9, wherein the adapting device further comprises at least an additional communication interface for expanding the functionality of the base station.

11. The system as claimed in claim 9, wherein the adapting device is implemented to have a dock structure for receiving the base station.

12. The system as claimed in claim 1, wherein the base station displays the waveforms of the received physiological signals.

13. The system as claimed in claim 1, wherein the base station further analyzes the received physiological signals, and when the analysis result is matched to a preset condition, and the base station sends out a warning signal to notify an operator in front of the remote computer device.

14. The system as claimed in claim 13, wherein the preset condition is configured by the operator through the base station and/or the remote computer device.

15. The system as claimed in claim 1, wherein the base station further comprises an light sensor, for sensing the changes of light in the environment.

16. The system as claimed in claim 1, wherein the remote computer device controls/configures the wireless physiological signal acquiring devices by means of a software embedded therein and the base station.

17. The system as claimed in claim 1, wherein the sensing element of the wireless physiological signal acquiring device is one or more selected from a group consisting of: a flow sensor, a thermistor, a snore sensor, respiratory effort belts, EEG electrodes, EOG electrodes, ECG electrodes, EMG electrodes, an oximeter, and a position sensor.

18. The system as claimed in claim 17, wherein the position sensor is built in at least one of the wireless physiological signal acquiring devices.

19. The system as claimed in claim 1, wherein the sensing elements are connected to the wireless physiological signal acquiring devices by a direct connection or via a connection mechanism.

20. The system as claimed in claim 19, wherein the connection mechanism is achieved by a flat-typed connector.

21. The system as claimed in claim 19, wherein the connection mechanism is implemented to connect multiple sensing elements to one wireless physiological signal acquiring device at the same time.
